(19) United States
(12) Patent Application Publication
(10) Pub. No.: US 2019/0175430 A1
(43) Pub. Date: Jun. 13, 2019

(54) ALL-IN-ONE SUSPENDABLE FABRIC URINAL HOLDER/COVER

(71) Applicant: Harriet B. Mentzer, Druimmond, MT (US)

(72) Inventor: Harriet B. Mentzer, Druimmond, MT (US)

(21) Appl. No.: 16/350,533

(22) Filed: Nov. 29, 2018

Related U.S. Application Data
(60) Provisional application No. 62/708,438, filed on Dec. 11, 2017.

(51) Int. Cl. A61G 9/00; A61F 5/455

(52) U.S. Cl. CPC A61G 9/006; A61F 5/4556

(57) ABSTRACT

The present invention is a bedside or chairside urinal holder/cover made entirely of durable washable fabric. Fabric pieces are sewn to conform to the shape of a urinal. Three sides of the holder/cover are tall enough to cover the urinal to the lid. The fourth side is shorter. Its semi-circular opening allows easy access to a portable urinal's handle. Permanently stitched to the top rim of the holder/cover is a closed-end loop, also made of durable washable fabric. The loop may be laid over the top of a bedrail or chair arm and the body of the holder/cover pulled through this loop to securely fasten it in place. The holder/cover cannot accidentally come unfastened, hides urinal contents, and gives an aesthetically pleasing appearance. It can be laundered and stored with other linens until it is put back into service. It takes little space and has a long lifespan.

### RELATED APPLICATIONS

This application claims the benefit of U.S. Provisional Patent Application No. 62/708,438 filed on Dec. 11, 2017.

### BACKGROUND OF THE INVENTION

The present invention relates to the field of patient care apparatus and more particularly to methods of providing easy access to portable urinals for patient use.

Patients in hospitals or nursing facilities often have a urinal provided for them for use at the bedside or while seated in a chair. As patients want them accessible, these urinals often are placed on the bedside table. This is unsightly and unsanitary. Also, some urinals are designed to hang over the bedrail. These, however, have a tendency to come loose and fall after even a slight sideways bump. A more attractive, more sanitary and secure method for keeping bedside urinals handy is needed.

### BRIEF SUMMARY OF THE INVENTION

An all-in-one suspendable fabric urinal holder/cover is made from materials that are easy to launder. One option is a polyester/cotton fabric blend. The urinal holder/cover, made in the shape of a portable urinal, has a permanently attached strap that loops over the railing of a bed or the arm of a chair. The body of the all-in-one is drawn through the loop to suspend it securely from the bedrail or chair arm. One side goes toward the head of the bed. The fabric on this side is tall enough to cover the urinal to its lid. The side toward the foot of the bed is shorter, allowing the handle of the urinal to be easily accessed.

### BRIEF DESCRIPTION OF THE SEVERAL VIEWS OF THE DRAWINGS

FIG. 1—a urinal

FIG. 2—an all-in-one suspendable fabric urinal holder/cover

FIG. 3—an all-in-one suspendable fabric urinal holder/cover in use, suspended from the bed rail with urinal mostly concealed yet easily accessible

FIG. 4—pattern for making the main body of the all-in-one suspendable fabric urinal holder/cover

### DETAILED DESCRIPTION OF THE INVENTION

Besides providing an attractive, sanitary method for keeping portable urinals handy, this invention holds several other unique advantages for patients and their caregivers. This invention (a) cannot unintentionally come unfastened, causing a half-full urinal to fall and spill its contents; (b) can be completely sanitized in the laundry; (c) reused many times; (d) requires less space for storage than other holders on the market; and, (e) because it is lightweight, collapsible and foldable, shipping costs are lower.

The following information illustrates the general principles of this invention by focusing on items shown in the drawings. The measurements mentioned are to fit one style of portable urinal and can be adjusted for other styles or sizes. The possible addition of a second strap, not illustrated here, allows the invention to be utilized on either side of the bed or chair in a position that provides added convenience for patient use.

Number 10 represents a portable urinal, which is not included in the invention but is shown to represent how it fits in the all-in-one suspendable fabric urinal holder/cover. A typical urinal is approximately 18 cm or 7 inches tall and bends at the neck for 8 cm or 3 more inches to the lid. The bottom of the urinal measures approximately 8 cm or 3 inches×9 cm or 3.5 inches, and the lid is approximately 8 cm or 3 inches in diameter. At its widest part, the urinal's circumference is approximately 33 cm or 13 inches.

Number 12 is a closed-end loop of fabric, permanently attached at the urinal holder/cover's upper edge. A typical loop measures approximately 16 mm or 0.625 inches wide by 41 cm or 16 inches long.

Number 14 represents the cutout that allows the urinal handle 10-2 to be positioned outside the holder/cover. An opening that is approximately 7 cm or 2.63 inches wide by 7 cm or 2.75 inches deep provides accessibility to the handle of the urinal for putting the body of the urinal in or out of the all-in-one suspendable fabric urinal holder/cover.

Number 16 represents the main body of an all-in-one suspendable fabric urinal holder/cover. The preferred embodiment is about 9 cm or 3.5 inches×10 cm or 4 inches at the bottom, and just over 25 cm or 10 inches tall. Its contours mimic the contours of the urinal.

Number 16-1 represents the all-in-one suspendable fabric urinal holder/cover's side that has the opening for the urinal. The measurement for this side, from the holder's base to the fabric's edge that serves as a hanger for the urinal's handle, is approximately 15 cm or 6 inches.

Number 16-2 represents the measurements of the bottom of a typical all-in-one suspendable fabric urinal holder/cover, approximately 9 cm or 3.5 inches×10 cm or 4 inches. This bottom piece may or may not incorporate washable plastic or interfacing to help it maintain its shape.

Number 16-3 represents each of the two sides of the all-in-one suspendable fabric urinal holder/cover that have a curve at the neck to fit the urinal. The overall height of each side measures approximately 27 cm or 10.5 inches, and width at the base measures approximately 10 cm or 4 inches.

Number 16-4 represents the back side of an all-in-one suspendable fabric urinal holder/cover. Once stitched, this side may have a height of approximately 27 cm or 10.5 inches and a width of 9 cm or 3½ inches.

Number 18 demonstrates how the closed-end loop is used to fasten the all-in-one suspendable fabric urinal holder/cover to a bedrail 20. The same technique may be used in hanging the invention from the arm of a chair.

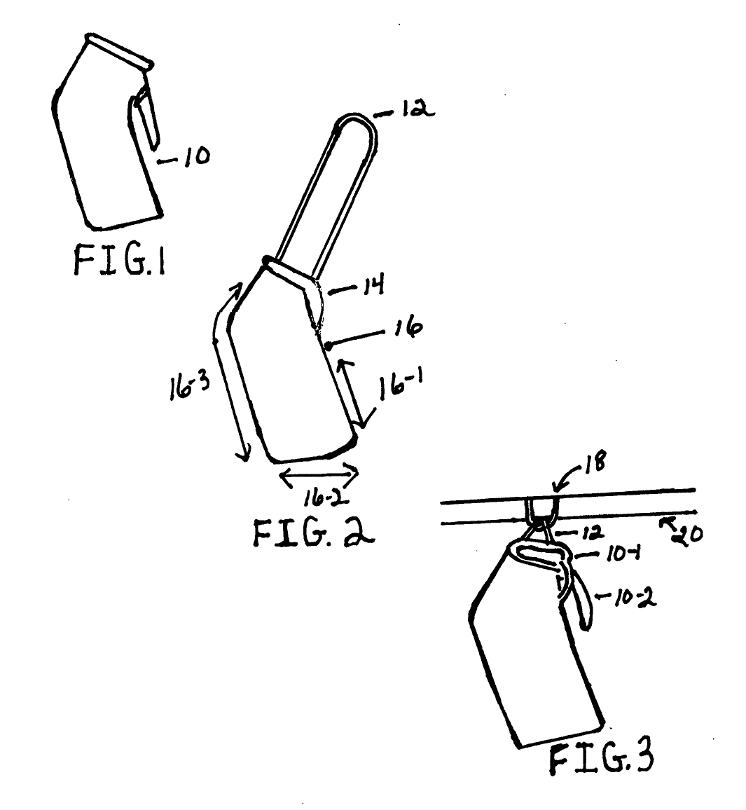

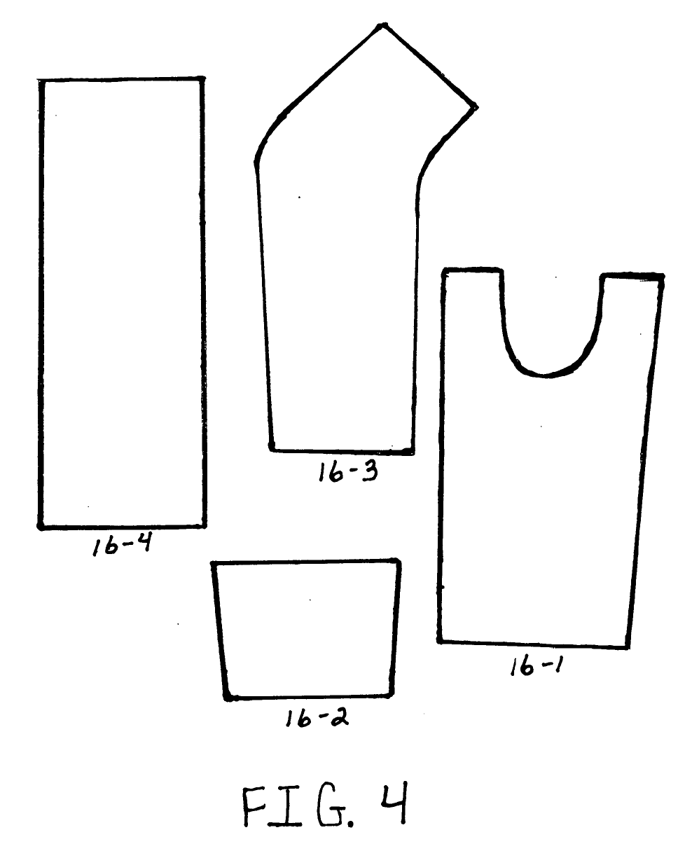

Number 10-2 and Number 10-1, FIG. 3, are illustrated for visual purposes only and show the handle 10-2 of a typical male urinal 10 with its lid 10-1 in place, inconspicuously awaiting use or content disposal.

In a preferred embodiment, the rim at the top of the all-in-one suspendable fabric urinal holder/cover is formed from 22.2 mm or 0.875-inch polyester/cotton blend double fold bias tape quilt binding lined with interfacing. This binding also is used to make the closed-end loop that is permanently stitched to the holder/cover's rim.

Instructions for using the pattern shown in FIG. 3 to make an all-in-one suspendable fabric urinal cover are as follows:
1. Use the pattern FIG. 4 to cut out fabric pieces. Fabric of durable polyester/cotton blend is preferred. Cut one piece for each segment of the pattern except as follows:
   Cut two bottom pieces 16-2 of fabric, and one bottom piece of fusible interfacing made of polyester. Also cut two of 16-3.
2. Place the interfacing between the two pieces of 16-2 and iron to fuse. Sew from corner to corner, making an X.
3. Sew binding to the curved area of 16-1.
4. Attach the two 16-3 pieces to 16-4 as follows:
   For one 16-3 piece, stitch the side with an outer curve to the long edge of 16-4, making ¼-inch seams. Repeat to attach the other 16-3 piece to the opposite edge of 16-4. Serge to finish edges. Press seams to center and top stitch.
5. Sew 16-1 on each side to the inner curved sides of 16-3, which completes the joining of the all-in-one suspendable fabric urinal holder/cover's front, back, and sides. Serge to finish.
6. Sew the completed bottom piece 16-2 to the bottom edge of the completed front, back, and sides. Serge to finish.
7. Cut one piece of interfacing that measures approximately 28 cm or 11 inches by 1 cm or 0.38 inches. Fold 13 mm or 0.5 inches of fabric at top edge of the urinal holder/cover over the interfacing and press to fuse together. Zigzag stitch to help reinforce.
8. Cut a length of approximately 31 cm or 12 inches of binding. Turn the ends under 7 mm or 0.25 inch and stitch.
9. Apply binding to top of urinal holder/cover. Stitch to secure.
10. Turn urinal holder/cover right side out.
11. Cut a 51 cm or 20-inch piece of binding to form the closed-end loop. Optionally, cut two 51 cm or 20-inch lengths, one for each side. Stitch along both edges of the binding.
12. Securely sew both ends of a length of binding to one side of the rim on the urinal holder/cover's rim. Space the ends 5 to 8 cm or 2 to 3 inches apart. A second length of binding on the opposite side may be added to allow the completed invention to be utilized on either side of a hospital bed or chair.

What is claimed is:

1. An all-in-one suspendable fabric urinal holder/cover that cannot unintentionally come unfastened, causing a half-full urinal to fall and spill its contents.

2. An all-in-one suspendable fabric urinal holder/cover that can be completely sanitized in the laundry and stored with other linens until it is put back into service.

3. An all-in-one suspendable fabric urinal holder/cover that requires little space for storage.

4. An all-in-one suspendable fabric urinal holder/cover wholly made of durable fabric, giving it a long lifespan.

5. An all-in-one suspendable fabric urinal holder/cover that can be shipped at low cost because it is lightweight, collapsible, and foldable.

6. An all-in-one suspendable fabric urinal holder/cover that conceals urinal contents, giving an aesthetically pleasing appearance.
